(19) United States
(12) Patent Application Publication
(10) Pub. No.: US 2022/0233093 A1
(43) Pub. Date: Jul. 28, 2022

(54) SYSTEMS AND METHODS TO PROVIDE A PHYSICIAN INTERFACE THAT ENABLES A PHYSICIAN TO ASSESS ASTHMA OF A SUBJECT AND PROVIDE THERAPEUTIC FEEDBACK

(71) Applicant: AsthmaTek, Inc., Danville, CA (US)

(72) Inventor: Jemal Dean Zikria, Danville, CA (US)

(21) Appl. No.: 17/156,277

(22) Filed: Jan. 22, 2021

(51) Int. Cl. A61B 5/08; G16H 50/30; G16H 20/10; A61B 5/00; A61B 5/087; A61B 5/091

(52) U.S. Cl. CPC A61B 5/082; G16H 50/30; G16H 20/10; A61B 5/4833; A61B 2560/0242; A61B 5/486; A61B 5/0871; A61B 5/091; A61B 5/7475

(57) ABSTRACT

Systems and methods configured to provide a physician interface that enables a physician to assess asthma of a subject and provide therapeutic feedback are disclosed. Exemplary implementations may: obtain subject information for the subject including environmental conditions at or around a physical location of the subject, and results of remote diagnostic tests and physician-administered diagnostic tests for the subject; determine, based on the subject information, a condition assessment regarding the asthma of the subject; effectuate presentation of physician interface via a client computing platform associated with the physician, physician interface configured to convey the condition assessment and enable the physician to provide the therapeutic feedback based on the condition assessment; receive the therapeutic feedback via physician interface, wherein the therapeutic feedback includes adjustments to the specified therapy regime; and transmit the therapeutic feedback to a client computing platform associated with the subject for presentation to the subject.

### FIELD OF THE DISCLOSURE

The present disclosure relates to systems and methods configured to provide a physician interface that enables a physician to assess asthma of a subject and provide therapeutic feedback.

### BACKGROUND

Individuals with asthma may only monitor and treat for his/her asthma symptoms, conditions, and causes. However, the individuals often do not monitor their conditions and causes correctly nor treat their symptoms correctly without information directly from a physician.

### SUMMARY

One aspect of the present disclosure relates to a system configured to provide a physician interface that enables a physician to assess asthma of a subject and provide therapeutic feedback to the subject. Subject information informative of the asthma of the subject may be obtained to facilitate assessment of the asthma and its effect on the subject. Based on the subject information, a condition assessment may be determined. The condition assessment may determine how well the subject is complying with a specified therapy regime, how much the asthma is impeding on the subject and their breathing functionality, and potential adjustments to the specified regime to improve the asthma and its effects. Such condition assessment may be conveyed by a physician interface presented to the physician via their client computing platform. The physician interface may enable the physician to provide therapeutic feedback. The therapeutic feedback may be received and transmitted to a client computing platform of the subject for presentation. Therefore, the physician may treat the asthma of the subject appropriately based on a plurality of recent information.

One aspect of the present disclosure relates to a system configured to provide a physician interface that enables a physician to assess asthma of a subject and provide therapeutic feedback. The system may include one or more hardware processors configured by machine-readable instructions. Instruction components may include one or more of subject information component, condition assessment component, interface effectuation component, feedback component, and/or other instruction components.

The subject information component may be configured to obtain subject information for the subject. The subject information may include (i) environmental conditions at or around a physical location of the subject, (ii) results of remote diagnostic tests for the subject, (iii) physician-certified condition information including results to physician-administered diagnostic tests for the subject, and/or other information.

The condition assessment component may be configured to determine a condition assessment regarding the asthma of the subject. Determining the condition assessment may be based on the subject information. The condition assessment may include a compliance assessment indicating compliance of the subject with a specified therapy regime, a condition impact assessment indicating a present impact of asthma on the subject, a therapy regime assessment indicating potential adjustments to the specified therapy regime, and/or other information.

The interface effectuation component may be configured to effectuate presentation of physician interface via a client computing platform associated with the physician. Physician interface may be configured to convey the condition assessment and enable the physician to provide the therapeutic feedback based on the condition assessment.

The feedback component may be configured to receive the therapeutic feedback via physician interface. The therapeutic feedback includes adjustments to the specified therapy regime. The feedback component may be configured to transmit the therapeutic feedback to a client computing platform associated with the subject for presentation to the subject. The therapeutic feedback may be transmitted responsive to receipt of the therapeutic feedback.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to provide a physician interface that enables a physician to assess asthma of a subject and provide therapeutic feedback, in accordance with one or more implementations.

FIG. 2 illustrates a method to provide a physician interface that enables a physician to assess asthma of a subject and provide therapeutic feedback, in accordance with one or more implementations.

FIG. 3A-D illustrate example implementations of the system configured to provide a physician interface that enables a physician to assess asthma of a subject and provide therapeutic feedback, in accordance with one or more implementations.

### DETAILED DESCRIPTION

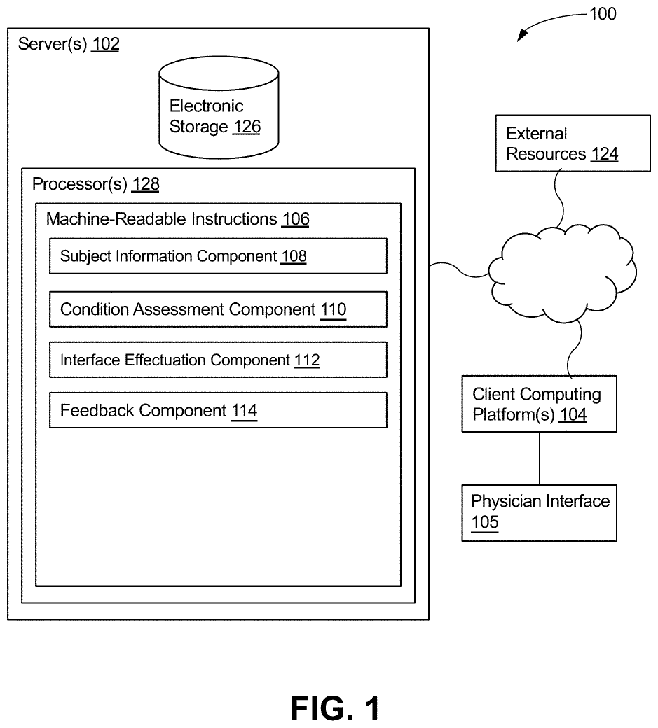

FIG. 1 illustrates a system 100 configured to provide a physician interface 105 that enables a physician to assess asthma of a subject and provide therapeutic feedback, in accordance with one or more implementations. Therefore, the physician may be informed during subject crises requiring medical intervention or guidance, office visits requiring retrospective analysis of asthma conditions and asthma management by the subject, remote monitoring of the subject that requires frequent monitoring and interactions with the subject, subject decisions related to crisis situations and remotely communicating with the physician, and/or other information.

In some implementations, system 100 may include one or more servers 102, client computing platforms 104, physician interfaces 105, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Subjects may access system 100 via client computing platform(s) 104.

Server(s) 102 may be configured by machine-readable instructions 106. Machine-readable instructions 106 may include one or more instruction components. The instruction components may include computer program components. The instruction components may include one or more of subject information component 108, condition assessment component 110, interface effectuation component 112, feedback component 114, and/or other instruction components.

Subject information component 108 may be configured to obtain subject information for the subject. The subject information may include (i) environmental conditions at or around a physical location of the subject, (ii) subject condition information (e.g., results of remote diagnostic tests for the subject), (iii) physician-certified condition information, and/or other subject information. As used herein, the term “physician” may refer to a doctor, a clinician, a nurse, a nurse practitioner, an asthma educator, a respiratory specialist, and/or or other authorized medical practitioner or healthcare providers.

The environmental conditions at or around the physical location may be indicated by location information related to the physical location of the subject over time. The physical location may refer to where the subject is over time. The subject may be at, around, or near the physical location and other various physical locations for an amount time (e.g., specified by the subject via a graphical user interface presented on client computing platform 104 associated with the subject). The location information may indicate the physical location of the subject. The physical location of the subject may be of a contextual environment of the subject. The contextual environment may be the immediate space surrounding the subject and of which the subject is working or otherwise breathing in. For example, the contextual environment may be outdoors or indoors. The environmental conditions may be based on the location information.

The location information may include geographical position system (GPS) coordinates, an internet protocol (IP) address, a geolocation based on client computing platform 104 of the subject and proximate cell towers, Wi-Fi location, and/or other information based on the subject's client computing platform 104 that indicates their physical location. In some implementations, the location information may be obtained in an ongoing manner to ensure accuracy of the physical location indicated. In some implementations, subject movements may be tracked using a geography-based transmitter on client computing device 104 associated with the subject.

The term “ongoing manner” as used herein may refer to continuing to perform an action (e.g., obtain) periodically (e.g., every 30 seconds, every minute, every hour, etc.) until receipt of an indication to terminate. The indication to terminate may include powering off client computing platform 104, charging one or more of a battery of client computing platform 104, subject input (e.g., via a virtual button), and/or other indications of termination. For example, the subject may move 7 miles from an initial physical location indicated by the location information and the location information should be obtained again to indicate a more accurate physical location of the subject.

In some implementations, the location information may include location input manually entered by the subject to indicate their physical location or one or more future physical locations. For example, the location input may include GPS coordinates, a street address, a zip code, a city, the contextual environment (e.g., indoors or outdoors) and/or other location information. In some implementations, the amount of time, a start time, and/or an end time that the subject is at, around, or near the physical location may be manually input by the subject. For example, the subject may input a start time of 5 PM for 3 hours, or 5 PM-8 PM.

By way of non-limiting example, the environmental conditions may include values to one or more of pollen levels, weather, an elevation, mold, dust and dander, an air quality index, and/or other environmental conditions. In some implementations, the values may be numerical and associated with units of measure. In some implementations, the values may be non-numerical and generally characterize the respective environmental condition (e.g., good, okay, low, medium, moderate, fair, high, poor, extreme). The values of pollen levels may indicate how much of tree pollen, grass pollen, weed pollen, flower pollen, and/or other pollen is present in air. The values to the weather may include an outdoor temperature, precipitation, humidity, and/or other values indicative of the weather at or around the physical location. The values to the mold may indicate an amount of mold spores in a cubic meter of the air, or other measurement, at or around the physical location. The values of the dust and dander may indicate how much dust and dander from animal fur, hair, feathers, and/or other sources are in an indoor area and/or outdoor area. In some implementations, the values of the dust and dander may be based on the values to the weather and/or other environmental conditions that may exacerbate the dust and dander or may not. The values to the air quality index may indicate how polluted the air at the physical location is. In some implementations, the air may be polluted with the pollen, the dust and dander, the mold spores, and/or other pollutants.

In some implementations, the location information may include future physical locations and/or the contextual environments of the subject. The future physical locations and/or the contextual environments may be based on a calendar, to-do lists, and/or other information that subject information component 108 has access to or based on the location input.

In some implementations, the environmental conditions that are based on the location information may be obtained (e.g., from weather systems) by subject information component 108. In some implementations, subject information component 108 may be configured to obtain the location information and determine the environmental conditions based on the location information obtained. Therefore, the environmental conditions for the future physical locations may be obtained or determined by subject information component 108.

In some implementations, the contextual environment may be the indoors (e.g., living room, office). In some implementations, the value to the air quality index may be based on air quality information from one or more digital air purification systems proximate to the subject in the indoor area. The one or more digital air purification systems may be proximate to the subject in the contextual environment, i.e., the indoor area, of the subject. The air quality information may convey ambient air quality measured in the contextual environment of the subject. The digital air purification systems may measure, calculate, derive from, or otherwise determine values indicative of quantities of dust and dander, mold, pollen level, and/or other information in the indoors.

In some implementations, the contextual environment may be the outdoors (i.e., the subject is outdoors). In some implementations, the value to the air quality index may be based on air quality information obtained from the external resources 124 for the outdoors. External resources 124 may include satellite systems that collect information about particles in the air, the dust and dander, the mold, the pollen level, and/or other external resources 124 that collect and/or provide air quality information for the outdoors.

In some implementations, the air quality information (obtained from the digital air purification system or external resources 124) may be associated with a particular time or period of time. In some implementations, the air quality information may be obtained in an ongoing manner. In some implementations, the air quality information may be obtained at a reoccurring predefined time and may convey the ambient air quality of past times or periods of time.

Thus, in some implementations, the values to the air quality index may be determined or otherwise derived from the air quality information obtained. In some implementations, subject information component 108 may be configured to obtain the values to the air quality index directly from and determined by external resources 124, the digital air purification systems, and/or other sources.

The subject condition information may define remote diagnostic information, subject health information, and/or other information. The subject condition information may include information related to asthma and general health of the subject. The remote diagnostic information may include past and most current results to remote diagnostic tests and medication administrators that the subject may conduct himself/herself as opposed to a clinic, doctor's office, or other medical office. The remote diagnostic tests and medication administrators may include a remote fractional exhaled nitric oxide (FeNO) test, a remote spirometry test, a remote peak flow test, a digital inhaler, and/or other remote diagnostic tests and medication administrators. The results may be associated with a date and/or time at which a given test or medication administration occurred. In some implementations, subject information component 108 may be configured to store the subject condition information to electronic storage 126, a cloud storage, and/or other storage. The subject condition information may be stored to electronic storage 126 indefinitely or for a predefined period of time. The period of time may be defined by the physician, the subject, fixed by system 100, and/or other individual.

A result to the remote FeNO test may determine an amount of lung inflammation of the subject. A result to the remote spirometry test may indicate how much air lungs of the subject is able to breathe in and out and how easily and fast the lungs are able to exhale the air. A result of the remote peak flow test may determine how fast the lungs are able to forcefully exhale after a full inhalation of air. The digital inhaler may indicate correctness of inhalation techniques of medication administered by the digital inhaler, frequency of the administrations, and/or other information related to medication administrations/consumptions via the digital inhaler. For example, the frequency of the administrations may indicate whether the patient is using the digital inhaler enough or too often. For example, the correctness of inhalation techniques may indicate whether the subject is positioning the digital inhaler at a correct angle during administration, shaking the digital inhaler enough or too much prior to use, and/or other correctness of inhalation techniques.

The subject health information may include values for one or more of cardiovascular parameters, sleep parameters, activity parameters, diet parameters, and/or other parameters. The subject health information (i.e., parameter included thereof) may specify measurable, recordable, and/or determined information. The values corresponding to the parameters included in the subject health information may be a particular value, numerical or non-numerical, that characterizes the subject.

By way of non-limiting example, the cardiovascular parameters may include a heart rate, a resting heart rate of the subject, a respiratory rate of the subject, blood pressure of the subject, oxygen saturation of the subject, heart variability, heartbeat strength, heartbeat rhythm, and/or other cardiovascular parameters. By way of non-limiting example, the sleep parameters may include hours of sleep, sleep quality, motion during sleep, breathing patterns during sleep, and/or other sleep parameters. By way of non-limiting example, the activity parameters may include minutes exercised; distance walked, ran, cycled, and/or swam; speed of the walk, the run, the cycle, and the swim; inclination of the walk, the run, and the cycled; acceleration, time standing, and/or other activity parameters. By way of non-limiting example, diet parameters may include caloric intake, weight, weight goal, height, body mass index (BMI), various vitamin intake, food restrictions, food intake, liquid intake, substance intake, and/or other diet parameters. Some of the subject health information may be obtained from one or more wearable devices worn by the subject. For example, the wearable devices may include an orientation sensor, a location sensor, a pressure sensor, a temperature sensor, a light sensor, an audio sensor, cardiovascular sensors, and/or other sensors. For example, the cardiovascular sensors may include an electrodermal activity (EDA), an electrocardiography (EKG or ECG) sensor, a blood volume pulse (BVP) sensor, a respiration sensor, a blood pressure sensor, and/or other cardiovascular sensors. It is to be expressly understood, however, that the subject health information obtained and the sensors that facilitation the obtainment are for the purpose of illustration and description only and are not intended as a definition of the limits of this disclosure.

In some implementations, subject information component 108 may be configured to receive manual subject condition information (i.e., remote diagnostic information, subject health information) the from client computing platform 104 associated with the subject described in FIG. 1. The manual subject condition information may be the same as the subject condition information though manually input by the subject via a graphical user interface presented on their client computing platform 104.

The physician-certified condition information may include results to physician-administered diagnostic tests for the subject and scores to tests/questionnaires collected by the physician related to the asthma of the subject. The physician-certified condition information may include physician-supervised diagnostic information, physician-administered evaluations of the subject, and/or other information collected by the physician related to the subject.

The physician-supervised diagnostic information may include past and most current results to physician-administered diagnostic tests including a physician-administered FeNO test, a physician-administered spirometry test, a physician-administered peak flow test, and/or other physician-administered diagnostic tests. The physician-administered FeNO test, the physician-administered spirometry test, and the physician-administered peak flow test, may be the same as or similar to the remote FeNO test, the remote spirometry test, and the remote peak flow test, respectively, though administered/conducted by the physician. In some implementations, the tests that are physician-administered may be conducted at a medical office, for example, or other setting (e.g., home of subject during house call). In some implementations, the physician-certified condition information may be obtained from clinical systems (e.g., associated with the medical office, hospitals, etc.), laboratory systems, outpatient clinic information technology (IT) systems, and/or other medical information systems, and/or external resources 124.

The physician-administered evaluations may include scores and/or notes to an asthma control questionnaire (ACQ), a physician-administered asthma control test (ACT), and/or other physician-administered evaluations. The scores to the ACQ may measure change in the controlling of the asthma and adequacy of the controlling of the asthma by the subject at a point in time (i.e., how well the subject is following an asthma action plan (AAP) or specified therapy regime). The physician may administer or ask questions included in the ACQ to the subject. In some implementations, the ACQ may be generated based on a known asthma control questionnaire template. In some implementations, the ACQ may be generated by the physician. The physician-administered ACT may refer to questions related to controlling their asthma that the physician may administered/asked the subject. In some implementations, the ACT may be a known set of questions. In some implementations, the known set of questions may be modifiable by the physician. The physician may ask the ACT periodically such as once a week, once a day, subsequent to severe asthma symptoms, and/or other at times. The physician-certified condition information may be stored to cloud storage, electronic storage 126, and/or other storage.

Condition assessment component 110 may be configured to determine a condition assessment regarding the asthma of the subject and/or other information. Determining the condition assessment may be based on all or some of the subject information. The condition assessment may include a compliance assessment, a condition impact assessment, a therapy regime assessment, and/or other information.

The compliance assessment may indicate compliance of the subject with a specified therapy regime. The specified therapy regime may be related to managing and/or improving the impact of asthma on the subject. The specified therapy regime may be followed by or attempted to be followed by the subject to manage and/or improve the impact of asthma on the subject and their breathing functionality. By way of non-limiting example, the specified therapy regime may include: inhaled corticosteroid (ICS) therapy, short acting beta agonists (SABA), long acting beta agonists (LABA), zileuton (e.g., Zyflo), leukotriene receptor antagonists (LTRA), theophylline, omalizumab, other biologics for asthma, other asthma drug therapy, and/or other therapy.

By way of non-limiting example, the SABAs may include Asthavent® metered dose inhaler (MDI)/DP-Haler®/Revolizer® (Salbutamol), Berotec® MDI (Fenoterol), Venteze® MDI (Salbutamol), Ventolin® MDI/Accuhaler® (Salbutamol), and/or other SABAs. By way of non-limiting example, the LABAs may include Foratec DP-Haler®/Revolizer® (Formoterol), Oxis Turbuhaler® (Formoterol), Serevent® MDI/Accuhaler® (Salmeterol), and/or other LABAs. By way of non-limiting example, the other drug asthma therapies may include anticholinergics (i.e., a reliever), drug therapy combinations, preventers such as inhaled corticosteroids (ICS) and the leukotriene receptor antagonists, and/or other drug therapies. The anticholinergics may include Atrovent® MDI (Ipratropium Bromide), Ipvent-40® MDI (Ipratropium Bromide), Spiriva Handihaler® (Tiotropium), and/or other anticholinergics. The drug therapy combinations may include DP-Haler®/Revolizer® (Budesonide+Formoterol), Seretide® MDI/Accuhaler® (Fluticasone+Salmeterol), Symbicord Turbuhaler® (Budesonide+Formoterol), and/or other drug therapy combinations. The inhaled corticosteroids may include Alvesco® MDI (Circlesonide), Beclate HFA® MDI (Beclomethasone), Budeflam DP-Haler®/Revolizer® (Budesonide), Budeflam HFA Gentle-Haler® (Budesonide), Flixotide® MDI/Accuhaler® (Fluticasone), Inflammide® MDI/Novolizer® (Budesonide), Pulmicort Turbuhaler® (Budesonide), QVAR® MDI (Beclomethasone), and/or other inhaled corticosteroids. The leukotriene receptor antagonists (LTRA) may include Singulair® tablets (Montelukast), and/or leukotriene receptor antagonists.

It is appreciated that the specified therapy regime listed is not intended to be limiting and is for illustrative purposes only as there may be various other therapies (e.g., non-medicinal exercises) specified for the subject to treat their asthma.

In some implementations, the compliance of the subject with the specific therapy regime may be indicated by a compliance value. The compliance value may be numerical (e.g., 1 out of 5) or non-numerical (e.g., A, B, C-, etc.) that characterizes how well the subject is following the specified therapy regime. In some implementations, the subject may specify portions of the specified therapy regime they have followed/completed and when. The compliance value may be determined based on the entry of the subject, the results to medication administrators indicating medication administration/consumption by the subject, and/or other subject information. In some implementations, compliance to the specified therapy regime may be measured as adherence (e.g., the voluntary cooperation of the patient in taking medications as prescribed, including timing, dosage, and frequency), “medical possession ratio” (MPR), and/or other compliance measurements.

The condition impact assessment may indicate a present impact of asthma on the subject and their breathing functionality. The present impact may be positive or negative. A positive present impact may indicate that the breathing functionality of the subject is not impeded by or less impeded by the asthma. For example, the positive present impact may be related to an improvement to the asthma of the subject and/or compliance with the specified therapy regime. A negative present impact may indicate that the breathing functionality of the subject is impeded by the asthma. For example, the negative present impact may be related to a lack of compliance with the specified therapy regime and/or the subject in physical locations or contextual environments that trigger asthma symptoms.

In some implementations, the subject information may include impact goals. In some implementations, the impact goals may be defined by the subject via their client computing platform 104 and/or the physician via physician interface 105. The impact goals may define a level of impact of the asthma on the subject (and their breathing functionality) to achieve and/or a level of impact of the asthma to maintain. The level of impact may be from an impact range. The impact range may be numerical or non-numerical. For example, the impact range may range from no symptoms, some symptoms, discomfort but tolerable, to difficulty breathing/functioning, and/or other levels. For example, the impact range may be 1-5, where a 1 refers to ease of breathing and/or no pain and 5 refers to difficulty breathing and pain. Thus, the present impact may be an impact value from the impact range.

In some implementations, the present impact may be determined based on changes in the subject information. In some implementations, condition assessment component 110 may be configured to determine the changes in the subject information. The changes in the subject information may indicate asthma symptoms for the subject, or a lack of asthma symptoms and good breathing functionality. Asthma symptoms may include shortness of breath, wheezing, coughing, chest pain, tightness of chest, rapid breathing, and/or other asthma symptoms.

By way of non-limiting example, changes in the subject information that indicate the subject has one or more of the asthma symptoms may include increases in one or more of the environmental conditions (e.g., the pollen levels, the elevation, the mold, the dust and dander, etc.), elevated values of the subject health information (e.g., elevated heart rate), low values of the subject health information, elevated results to the remote diagnostic tests (e.g., the remote FeNO test), low results to the remote diagnostic tests (e.g., the remote spirometry test), elevated results to the physician-administered diagnostic tests, low results to the physician-administered diagnostic tests, and/or other changes in the subject information.

To indicate the subject has a negative present impact from the asthma symptoms, one or more of the changes in the subject information may occur, a particular amount of the changes in the subject information may occur, one or more of the changes in the subject information may reach respective specific threshold values, one or more of the changes in the subject information may reach respective specific threshold values within a particular amount of time, and/or other various combinations of the changes and/or other changes in the subject information. In some implementations, the threshold values may be of a metric unit, or lack thereof, of the respective subject information. The threshold values may be known values that indicate the subject has asthma symptoms or may be defined by the physician, the subject, system 100, and/or other individuals. The particular amount of time may be defined by the physician, the subject, system 100, and/or other individuals. A particular amount of the subject information changing may be defined by the physician, the subject, system 100, and/or other individuals. As such, in some implementations, the condition impact assessment may define a source for the present impact of the asthma on the subject. The source may be the changes in the subject information.

The therapy regime assessment may indicate potential adjustments to the specified therapy regime. The potential adjustments may further the management and/or the improvement of asthma of the subject. For example, potential adjustments may include initiation/adjustment/termination of the ICS therapy, initiation/adjustment/termination of the SABAs, initiation/adjustment/termination of the LABAs, initiation/adjustment/termination of the zileuton, initiation/adjustment/termination of the LTRAs, initiation/adjustment/termination of the theophylline, initiation/adjustment/termination of the omalizumab, initiation/adjustment/termination of the other biologics for asthma, initiation/adjustment/termination of the other drug therapies, and/or other potential adjustments to the specified therapy regime.

In some implementations, condition assessment component 110 may be configured to determine the potential adjustments to the specified therapy regime based on one or more of the compliance assessment, a comparison of the present impact with the impact goals, the changes in the subject information, and/or other information. In some implementations, individual ones of the potential adjustments may be predetermined. That is, a given change in the subject information, a given compliance value, a given impact value, and/or other information may be associated with a particular potential adjustment. For example, a high impact value and a high compliance value indicating that the subject is complying with the specified therapy regime but has several asthma symptoms may be associated with adjusting (e.g., increasing) a dosage of the digital inhaler by 20%. For example, a high impact value and a low compliance value indicating that the subject is not complying with the specified therapy regime and has several asthma symptoms may be associated with causing daily reminders to be sent to the subject. The comparison of the present impact value and the impact goals may determine whether the subject is achieving the impact goals or not. In some implementations, no potential adjustments may be determined and thus the therapy regime may require no adjustments.

In some implementations, the potential adjustments may be determined and occur in a stepwise approach that incrementally steps up to increase dosages and/or add drug therapies or other therapies, and conversely may incrementally step down when appropriate (i.e., upon determination by condition assessment component 110). For example, the stepwise approach may step up beginning with the SABAs as needed; to a low-dose ICS; to a low-dose ICS with either one of a LABA, a LTRA, or Theophylline, or alternatively a medium lose ICS; to a medium-dose LABA; to a high-dose ICS with a LABA; to a high-dose ICS with a LABA and oral systemic Corticosteroids, and step down as appropriately determined by condition assessment component 110.

In some implementations, condition assessment component 110 may be configured to receive a physician indication to determine the condition assessment. In some implementations, the physician indication may be received upon the physician selecting a virtual button, for example, included on physician interface 105. The condition assessment may be determined responsive to receipt of the physician indication. In some implementations, the determination of the condition assessment may be defined by the physician to reoccur at a particular time. For example, the physician may define for the condition assessment to occur weekly every Friday, monthly every 1^{st} day of the month, or other time.

In some implementations, responsive to the changes in the subject information and/or the condition assessment, condition assessment component 110 may be configured to generate notifications. A notification may include the condition assessment, the potential adjustments, and/or other information. For example, the notification may be generated responsive to the compliance value of the compliance assessment reaching a threshold value that indicates the subject is complying well and include information related to the compliance value and/or other information. For example, the notification may be generated responsive to the impact value to the condition impact assessment reaching a threshold value that indicates the asthma is exceedingly impeding on the subject's breathing and include information related to the impact value and/or other information. In some implementations, absence of generation of a notification may indicate the subject is complying with the specified therapy regime, the asthma has a low impact on the subject's breathing functionality, or otherwise that the subject is managing their asthma well. The notifications may be conveyed to the physician by physician interface 105. In some implementations, the notification may include a pop-up window, a noise, haptics, and/or other notifications.

In some implementations, condition assessment component 110 may be configured to determine correlations between the subject condition information and the environmental conditions and/or between other information related to the subject and their asthma. That is, for example, a determined increase in elevation may correlate with a particular result to the remote FeNO test. Such correlation may imply that increased or high elevations affect the asthma of the subject as indicated by the particular result to the remote FeNO test. It will be appreciated that the description herein of “correlations” between the subject condition information and changes in environmental conditions which are positively correlated is not intended to be limiting, and that negative correlations the subject condition information and changes in environmental conditions are also contemplated, and may be included in the generic “correlations”. The determination of negative correlations may be made in cases where values of low humidity (i.e., very dry conditions) cause a high result to the remote FeNO test indicative of extreme lung inflammation.

The determined correlations may be conveyed to the physician by physician interface 105. In some implementations, the potential adjustments may be based on the determined correlations and the future physical locations of the subject. For example, the potential adjustment may be adjusting dosage of medication based on the correlation between the high elevations and the particular result to the remote FeNO test and upon determination that the subject will be at a high elevation soon.

Interface effectuation component 112 may be configured to effectuate presentation of physician interface 105 via client computing platform 104 associated with the physician. Physician interface 105 may be configured to convey the condition assessment, the subject information, the correlations determined, and/or other information related to the asthma of the subject. Physician interface 105 may be configured to enable the physician to provide the therapeutic feedback based on one or more of the condition assessment, the subject information, the correlations, and other information related to the subject. For example, the other information related to the subject may be stored in electronic storage 126. Physician interface 105 may include user interface elements. Individual user interface elements may be configured to be selected by the physician to cause presentation of particular information related to the subject (e.g., the condition assessment). Individual user interface elements may be configured to be selected or utilized by the physician to complete the ACQ, the physician-administered ACT, or other questionnaires. The user interface elements may be configured to facilitate physician interaction with physician interface 105, subject entry, and/or selection. By way of non-limiting illustration, the user interface elements may include one or more of text input fields, drop down menus, check boxes, display windows, virtual buttons, and/or other user interface elements.

In some implementations, physician interface 105 may convey the potential adjustments. The potential adjustments may be selectable (e.g., checkboxes) by the physician for transmission and presentation to client computing platform 104 associated with the subject as the therapeutic feedback. The information conveyed by physician interface 105 (e.g., the subject condition information, the condition assessment, the notifications, etc.), may allow for incremental increases in asthma health and reduction in catastrophic medical incidents of the subject.

Feedback component 114 may be configured to receive the therapeutic feedback via physician interface 105. The therapeutic feedback may include adjustments to the specified therapy regime, one or more messages, and/or other information. In some implementations, the adjustments may be manually entered by the physician. In some implementations, the adjustments to the specified therapy regime may be based on physician selection of the potential adjustments included in the therapy regime assessment. The one or more messages may be for the subject and related to the condition assessment. In some implementations, the physician may select particular user interface elements to enter a message and send the message. Thus, the messages may be transmitted to client computing platform 104 associated with the subject for presentation to the subject.

In some implementations, feedback component 114 may be configured to receive the specified therapy regime for the subject. The specified therapy regime may be received via physician interface 105 and entered by the physician. Thus, the specified therapy regime may be followed, or attempted to be followed, by the subject and may be the basis for at least the compliance assessment, the therapy regime assessment, or other condition assessment.

Feedback component 114 may be configured to transmit the therapeutic feedback to client computing platform 104 associated with the subject for presentation to the subject. The therapeutic feedback may be transmitted responsive to receipt of the therapeutic feedback. The therapeutic feedback may be transmitted over a network. In some implementations, the therapeutic feedback may be automatically formatted in accordance with a feedback format for the transmission. Subsequently, the formatted therapeutic feedback may be automatically transmitted to client computing platform 104 associated with the subject without physician input (e.g., selection of a transmission virtual button). The feedback format may be defined by the physician via physician interface 105 or predefined by system 100. The feedback format may be modified by the physician via physician interface 105 or fixed. In some implementations, upon selection of the particular user interface element to enter the message for the subject, the therapeutic feedback (i.e., the adjustments and/or the message) may be transmitted upon selection of the selection of a user interface element (e.g., send/transmit virtual button).

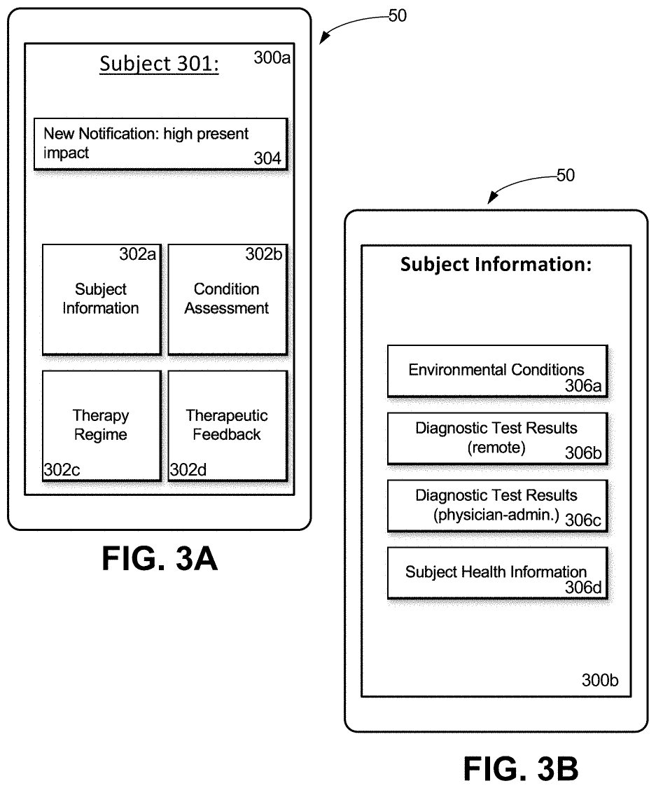

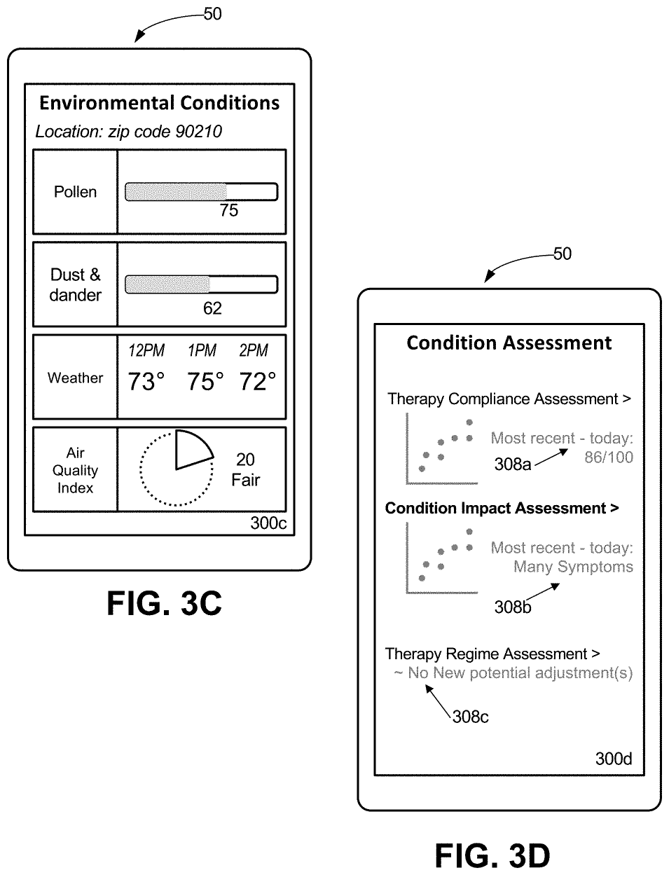

FIG. 3A-D illustrate example implementations, in accordance with one or more implementations. FIG. 3A illustrates a physician interface 300a on a smartphone 50. Physician interface 300a may include virtual buttons 302a, 302b, 302c, and 302d. Virtual button 302a-d may be selected by a physician to present information related to asthma of a subject 301. Virtual button 302a may represent subject information obtained (as described in FIG. 1) that may be presented upon selection of virtual button 302a. Virtual button 302b may represent a condition assessment (as described in FIG. 1) of subject 301 that may be presented upon selection of virtual button 302b. Virtual button 302c may represent a specified therapy regime (as described in FIG. 1) for subject 301 that may be presented upon selection of virtual button 302c and enable the physician to define or modify the specified therapy regime. Virtual button 302d may represent therapeutic feedback (as described in FIG. 1) for subject 301 from the physician that may be presented upon selection of virtual button 302d and enable the physician to define or modify the therapeutic feedback. Notifications, such as notification 304, may be conveyed on physician interface 300a. Notification 304 may notify the physician that subject 301 has a high present impact as indicated by the condition assessment (i.e., the condition impact assessment).

FIG. 3B illustrates physician interface 300b on smartphone 50, the same as smartphone 50 in FIG. 3A. Physician interface 300b may include virtual buttons 306a, 306b, 306c, and 306d that present environmental conditions, results of remote diagnostic tests, results to physician-administered diagnostics tests, and subject health information, respectively, upon selection (as described in FIG. 1). For example, FIG. 3C illustrates physician interface 300c on smartphone 50, the same as smartphone 50 in FIG. 3A-B. Physician interface 300c may present the environmental conditions obtained or determined including values to pollen, dust and dander, weather, and air quality index at the physical location (e.g., zip code 90210) of subject 301 (same as FIG. 3A).

FIG. 3D illustrates physician interface 300d on smartphone 50, the same as smartphone 50 in FIG. 3A-C. Physician interface 300d may present the condition assessment of subject 301. The condition assessment, as described in FIG. 1, may include information related to impact of asthma on the subject and the specified therapy regime. Physician interface 300d may include a preview value 308a of a therapy compliance assessment, preview value 308b of a condition impact assessment, and preview potential adjustments 308c from a therapy regime assessment. In some implementations, upon selection of preview value 308a, preview value 308b, and preview potential adjustments 308c, respective additional values and information related to such may be presented. For example, selection of preview value 308b may present information related to notification 304 from FIG. 3A.

Referring back to FIG. 1, in some implementations, server(s) 102, client computing platform(s) 104, and/or external resources 124 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 124 may be operatively linked via some other communication media.

A given client computing platform 104 may include one or more processors configured to execute computer program components. The computer program components may be configured to enable an expert or subject associated with the given client computing platform 104 to interface with system 100 and/or external resources 124, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 124 may include sources of information outside of system 100, external entities participating with system 100, and/or other resources. In some implementations, some or all of the functionality attributed herein to external resources 124 may be provided by resources included in system 100.

Server(s) 102 may include electronic storage 126, one or more processors 128, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 126 may comprise non-transitory storage media that electronically stores information. The electronic storage media of electronic storage 126 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 126 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 126 may include one or more virtual storage resources (e.g., cloud storage, a virtual private network, and/or other virtual storage resources). Electronic storage 126 may store software algorithms, information determined by processor(s) 128, information received from server(s) 102, information received from client computing platform(s) 104, and/or other information that enables server(s) 102 to function as described herein.

Processor(s) 128 may be configured to provide information processing capabilities in server(s) 102. As such, processor(s) 128 may include one or more of a digital processor, an analog processor, a digital circuit designed to process information, an analog circuit designed to process information, a state machine, and/or other mechanisms for electronically processing information. Although processor(s) 128 is shown in FIG. 1 as a single entity, this is for illustrative purposes only. In some implementations, processor(s) 128 may include a plurality of processing units. These processing units may be physically located within the same device, or processor(s) 128 may represent processing functionality of a plurality of devices operating in coordination. Processor(s) 128 may be configured to execute components 108, 110, 112, and/or 114, and/or other components. Processor(s) 128 may be configured to execute components 108, 110, 112, and/or 114, and/or other components by software; hardware; firmware; some combination of software, hardware, and/or firmware; and/or other mechanisms for configuring processing capabilities on processor(s) 128. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 128 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 128 may be configured to execute one or more additional components that may perform some or all of the functionality attributed below to one of components 108, 110, 112, and/or 114.

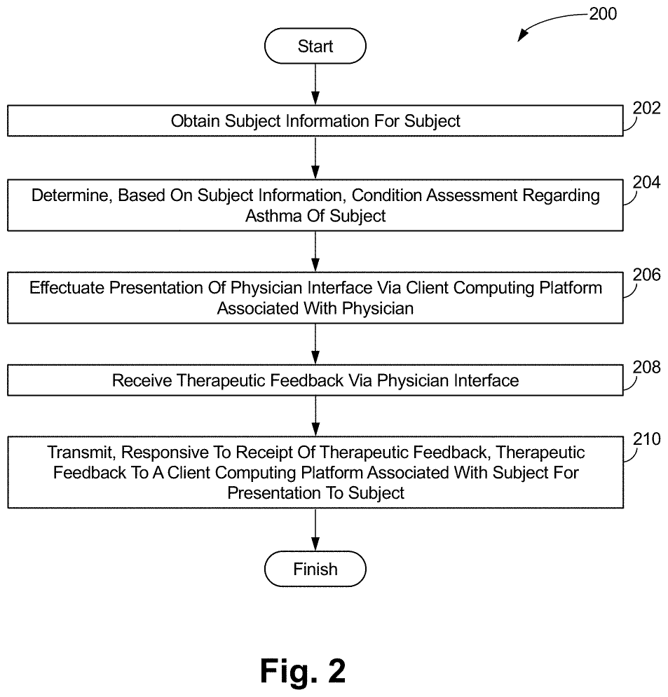

FIG. 2 illustrates a method 200 configured to provide a physician interface that enables a physician to assess asthma of a subject and provide therapeutic feedback, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include obtaining subject information for the subject. The subject information may include (i) environmental conditions at or around a physical location of the subject, (ii) results of remote diagnostic tests for the subject, and (iii) physician-certified condition information including results to physician-administered diagnostic tests for the subject. Operation 202 may be performed by one or more hardware processors configured by machine-readable instructions including a component that is the same as or similar to subject information component 108, in accordance with one or more implementations.

An operation 204 may include determining, based on the subject information, a condition assessment regarding the asthma of the subject. The condition assessment may include a compliance assessment indicating compliance of the subject with a specified therapy regime, a condition impact assessment indicating a present impact of asthma on the subject, and a therapy regime assessment indicating potential adjustments to the specified therapy regime. Operation 204 may be performed by one or more hardware processors configured by machine-readable instructions including a component that is the same as or similar to condition assessment component 110, in accordance with one or more implementations.

An operation 206 may include effectuating presentation of physician interface 105 via a client computing platform associated with the physician. Physician interface 105 may be configured to convey the condition assessment and enable the physician to provide the therapeutic feedback based on the condition assessment. Operation 206 may be performed by one or more hardware processors configured by machine-readable instructions including a component that is the same as or similar to interface effectuation component 112, in accordance with one or more implementations.

An operation 208 may include receiving the therapeutic feedback via physician interface 105. The therapeutic feedback may include adjustments to the specified therapy regime. Operation 208 may be performed by one or more hardware processors configured by machine-readable instructions including a component that is the same as or similar to feedback component 114, in accordance with one or more implementations.

An operation 210 may include transmitting, responsive to receipt of the therapeutic feedback, the therapeutic feedback to a client computing platform associated with the subject for presentation to the subject. Operation 210 may be performed by one or more hardware processors configured by machine-readable instructions including a component that is the same as or similar to feedback component 114, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to provide a physician interface that enables a physician to assess asthma of a subject and provide therapeutic feedback, the system comprising: one or more processors configured by machine-readable instructions to:
obtain subject information for the subject, wherein the subject information includes (i) environmental conditions at or around a physical location of the subject, (ii) results of remote diagnostic tests for the subject, and (iii) physician-certified condition information including results to physician-administered diagnostic tests for the subject;
determine, based on the subject information, a condition assessment regarding the asthma of the subject, wherein the condition assessment includes a compliance assessment indicating compliance of the subject with a specified therapy regime, a condition impact assessment indicating a present impact of asthma on the subject, and a therapy regime assessment indicating potential adjustments to the specified therapy regime;
effectuate presentation of the physician interface via a client computing platform associated with the physician, wherein the physician interface is configured to convey the condition assessment and enable the physician to provide the therapeutic feedback based on the condition assessment;
receive the therapeutic feedback via the physician interface, wherein the therapeutic feedback includes adjustments to the specified therapy regime; and
transmit, responsive to receipt of the therapeutic feedback, the therapeutic feedback to a client computing platform associated with the subject for presentation to the subject.

2. The system of claim 1, wherein the specified therapy regime is related to managing and/or improving the impact of asthma on the subject, wherein the one or more processors are further configured by the machine-readable instructions to:
receive, via the physician interface, the specified therapy regime for the subject.

3. The system of claim 1, wherein the physician interface conveys the subject information.

4. The system of claim 1, wherein the condition impact assessment defines a source for the present impact of the asthma on the subject.

5. The system of claim 1, wherein the one or more processors are further configured by the machine-readable instructions to:
determine the potential adjustments to the specified therapy regime based on one or more of the compliance assessment, and a comparison of the present impact with impact goals,
wherein the impact goals define a level of impact of the asthma on the subject to achieve and/or a level of impact of the asthma to maintain,
wherein the physician interface conveys the potential adjustments, and wherein the adjustments to the specified therapy regime are based on physician selection of the potential adjustments.

6. The system of claim 5, wherein individual ones of the potential adjustments are predetermined.

7. The system of claim 1, wherein the therapeutic feedback is automatically formatted in accordance with a feedback format for the transmission.

8. The system of claim 1, wherein the therapeutic feedback includes a message for the subject related to the condition assessment so that the message is transmitted to the client computing platform associated with the subject.

9. The system of claim 1, wherein the one or more processors are further configured by the machine-readable instructions to:
receive a physician indication to determine the condition assessment, wherein the condition assessment is determined responsive to receipt of the physician indication.

10. The system of claim 1, wherein the remote diagnostic tests include a remote fractional exhaled nitric oxide (FeNO) test, a remote spirometry test, and a remote peak flow test, and wherein the physician-administered diagnostic tests include a physician-administered FeNO test, a physician-administered spirometry test, and a physician-administered peak flow test.

11. A method to provide a physician interface that enables a physician to assess asthma of a subject and provide therapeutic feedback, the method comprising:
obtaining subject information for the subject, wherein the subject information includes (i) environmental conditions at or around a physical location of the subject, (ii) results of remote diagnostic tests for the subject, and (iii) physician-certified condition information including results to physician-administered diagnostic tests for the subject;
determining, based on the subject information, a condition assessment regarding the asthma of the subject, wherein the condition assessment includes a compliance assessment indicating compliance of the subject with a specified therapy regime, a condition impact assessment indicating a present impact of asthma on the subject, and a therapy regime assessment indicating potential adjustments to the specified therapy regime;
effectuating presentation of the physician interface via a client computing platform associated with the physician, wherein the physician interface is configured to convey the condition assessment and enable the physician to provide the therapeutic feedback based on the condition assessment;
receiving the therapeutic feedback via the physician interface, wherein the therapeutic feedback includes adjustments to the specified therapy regime; and
transmitting, responsive to receipt of the therapeutic feedback, the therapeutic feedback to a client computing platform associated with the subject for presentation to the subject.

12. The method of claim 11, wherein the specified therapy regime is related to managing and/or improving the impact of asthma on the subject, further comprising:
receiving, via the physician interface, the specified therapy regime for the subject.

13. The method of claim 11, wherein the physician interface conveys the subject information.

14. The method of claim 11, wherein the condition impact assessment defines a source for the present impact of the asthma on the subject.

15. The method of claim 11, further comprising:
determining the potential adjustments to the specified therapy regime based on one or more of the compliance assessment, and a comparison of the present impact with impact goals,
wherein the impact goals define a level of impact of the asthma on the subject to achieve and/or a level of impact of the asthma to maintain,
wherein the physician interface conveys the potential adjustments, and wherein the adjustments to the specified therapy regime are based on physician selection of the potential adjustments.

16. The method of claim 15, wherein individual ones of the potential adjustments are predetermined.

17. The method of claim 11, wherein the therapeutic feedback is automatically formatted in accordance with a feedback format for the transmission.

18. The method of claim 11, wherein the therapeutic feedback includes a message for the subject related to the condition assessment so that the message is transmitted to the client computing platform associated with the subject.

19. The method of claim 11, further comprising:
receiving a physician indication to determine the condition assessment, wherein the condition assessment is determined responsive to receipt of the physician indication.

20. The method of claim 11, wherein the remote diagnostic tests include a remote fractional exhaled nitric oxide (FeNO) test, a remote spirometry test, and a remote peak flow test, and wherein the physician-administered diagnostic tests include a physician-administered FeNO test, a physician-administered spirometry test, and a physician-administered peak flow test.
